(19) United States
(12) Patent Application Publication
(10) Pub. No.: US 2016/0067668 A1
(43) Pub. Date: Mar. 10, 2016

(54) COST-EFFECTIVE MATERIALS FOR PROCESS UNITS USING ACIDIC IONIC LIQUIDS

(71) Applicants: Hye Kyung Cho Timken, Albany, CA (US); Kevin Lewis Ganschow, Danville, CA (US); Andrew Nissan, Richmond, CA (US); Christine Marie Phillips, Pleasant Hill, CA (US)

(72) Inventors: Hye Kyung Cho Timken, Albany, CA (US); Kevin Lewis Ganschow, Danville, CA (US); Andrew Nissan, Richmond, CA (US); Christine Marie Phillips, Pleasant Hill, CA (US)

(73) Assignee: Chevron U.S.A. Inc., San Ramon, CA (US)

(21) Appl. No.: 14/481,145

(22) Filed: Sep. 9, 2014

(51) Int. Cl. B01J 19/02; C07C 2/62

(52) U.S. Cl. CPC B01J 19/02; C07C 2/62; C07C 2531/26; B01J 2219/0286

(57) ABSTRACT

We provide an apparatus for performing a hydrocarbon conversion or for handling of an output of the hydrocarbon conversion, comprising: a bare metal alloy, wherein the bare metal alloy comprises: from 15.1 to 49 wt % nickel, from 2.3 to 10 wt % molybdenum, from 0.00 to 2.95 wt % copper, and 20 to 59 wt % iron; wherein the bare metal alloy exhibits a corrosion rate less than 0.07 mm/year when performing the hydrocarbon conversion or handling the output of the hydrocarbon conversion; and wherein the hydrocarbon conversion is performed using an acidic ionic liquid. We also provide a process for using the apparatus.

### TECHNICAL FIELD

This application is directed to more cost-effective materials of construction for process units utilizing acidic ionic liquids and for processes using them.

### BACKGROUND

Lower cost and more easily obtainable alloys for use with acidic ionic liquids are needed. New processes for using these materials with acidic ionic liquids are also needed.

### SUMMARY

This application provides an apparatus for performing a hydrocarbon conversion or for handling of an output of the hydrocarbon conversion, comprising: a bare metal alloy, wherein the bare metal alloy comprises: from 15.1 to 49 wt % nickel, from 2.3 to 10 wt % molybdenum, from 0.00 to 2.95 wt % copper, and 20 to 59 wt % iron; wherein the bare metal alloy exhibits a corrosion rate less than 0.07 mm/year when performing the hydrocarbon conversion or handling the output of the hydrocarbon conversion; and wherein the hydrocarbon conversion is performed using an acidic ionic liquid.

This application also provides a process for performing a hydrocarbon conversion or for handling of an output of the hydrocarbon conversion, comprising using an apparatus comprising a bare metal alloy, wherein the bare metal alloy comprises: from 15.1 to 49 wt % nickel, from 2.3 to 10 wt % molybdenum, from 0.00 to 2.95 wt % copper, and 20 to 59 wt % iron; wherein the bare metal alloy exhibits a corrosion rate less than 0.07 mm/year when performing the hydrocarbon conversion or handling the output of the hydrocarbon conversion; and wherein the hydrocarbon conversion is performed using an acidic ionic liquid.

The present invention may suitably comprise, consist of, or consist essentially of, the elements in the claims, as described herein.

### BRIEF DESCRIPTION OF THE DRAWINGS

FIG. 1 is a graph of the corrosion rates of various metals in alkylation experiments.

FIG. 2 is a graph of the corrosion rates of titanium alloys and selected nickel alloys in alkylation experiments.

FIG. 3 is a graph of the wt % nickel vs. corrosion rates in alkylation experiments.

FIG. 4 is a graph of the wt % molybdenum vs. corrosion rates in alkylation experiments.

FIG. 5 is a graph of the wt % chromium vs. corrosion rates in alkylation experiments.

FIG. 6 is a graph of the combined wt % nickel+molybdenum+chromium vs. corrosion rates in alkylation experiments.

### GLOSSARY

“Corrosion” refers to the gradual destruction of materials (usually metals) by chemical reaction with their environment. Corrosion can be concentrated locally to form a pit or crack, or it can extend across a wide area more or less uniformly corroding the surface. Because corrosion is a diffusion-controlled process, it typically occurs on exposed surfaces.

“Corrosion rate” refers to the calculated value of either millimeters/year or mils/year, based on metal weight loss of a corrosion coupon, due to corrosion over a period of time.

“Bare metal alloy” refers to a metal comprising a mixture of elements and that is not coated with a non-metallic material, or other material applied to it prior to exposure to a corrosive agent, which prevents it from directly contacting a corrosive vapor or liquid.

“Acidic ionic liquid” refers to materials consisting entirely of ions, that can donate a proton or accept an electron pair in reactions, and that are liquid below 100° C.

“Stainless steel” is a steel alloy with a minimum of 10.5% chromium content by mass. Stainless steel does not readily corrode, rust or stain with water as ordinary steel does. There are different grades and surface finishes of stainless steel to suit the environment the alloy must endure. Stainless steel is used where both the properties of steel and corrosion resistance are required.

“Stress corrosion cracking” (SCC) refers to the growth of crack formation in a corrosive environment. SCC can lead to unexpected sudden failure of normally ductile metals subjected to a tensile stress, especially at elevated temperatures. SCC is highly chemically specific in that certain alloys are likely to undergo SCC only when exposed to a small number of chemical environments. The chemical environment that causes SCC for a given alloy is often one which is only mildly corrosive to the metal otherwise. Hence, metal parts with severe SCC can appear bright and shiny, while being filled with microscopic cracks. This factor makes it common for SCC to go undetected prior to failure. SCC often progresses rapidly, and is more common among alloys than pure metals. The specific environment is of crucial importance, and only very small concentrations of certain highly active chemicals are needed to produce catastrophic cracking, often leading to devastating and unexpected failure.

### DETAILED DESCRIPTION

### Apparatus:

Apparatuses that are used for performing hydrocarbon conversions or are used for handling an output of the hydrocarbon conversion can be exposed to an acidic ionic liquid, co-catalysts used with the acidic ionic liquid, or by products of the hydrocarbon conversion using the acidic ionic liquid. The acidic ionic liquid, co-catalysts, or by products produced during a hydrocarbon conversion using an acidic ionic liquid can contribute to corrosion of the apparatus. Examples of these types of apparatuses include reactors, conduits, fittings, heat exchangers, phase separators, distillation units, and combinations thereof.

Examples of reactors include continuously stirred tank reactors, fixed bed reactors, nozzles, motionless mixers, and pressure vessels.

Examples of conduits can include pipes, tubes, and flexible equipment designed to conduct a gas or liquid. In one embodiment, the conduit is a pipe. Fittings can include, for example, valves, elbows, unions, couplings, reducers, olets, tees, crosses, caps, plugs, nipples, injectors, barbs, gaskets, and the like. In one embodiment, the fitting is a valve, an elbow, or a coupling.

A heat exchanger is a piece of equipment built for efficient heat transfer from one medium to another. The media in the heat exchanger may be separated by a solid wall to prevent mixing or they may be in direct contact. Examples of types of heat exchangers include fluid, electric heating, double pipe, shell & tube, plate, plate & shell, adiabatic wheel, plate fin, pillow plate, waste heat recovery, dynamic scraped surface, and phase change.

Phase separators can include gas/liquid separators, liquid/liquid separators, and solid/liquid separators. Phase separators can use one of more of the following methods to achieve separation: density difference, gravity, impingement, change of flow direction, change of flow velocity, coalescence, centrifugal force, cyclonic action, filtration, agitation, heat, and combinations thereof. In one embodiment, the phase separator is a gas/liquid separator or a liquid/liquid separator.

Distillation units separate the component substances from a liquid mixture by selective vaporization and condensation. A distillation unit may produce essentially complete separation (nearly pure components), or it may produce a partial separation that increases the concentration of selected components of the mixture. An example of an apparatus using a distillation unit in the presence of an acidic ionic liquid to perform ionic liquid catalyzed hydrocarbon conversion is described in US Patent Pub. No. 20110319694A1.

In one embodiment, the apparatus is configured to produce an alkylate gasoline blending component, a distillate fuel, a base oil, or combinations thereof. Examples of these types of apparatuses are described in U.S. Patent Publication Numbers US20140134065A1, US20140066678A1, US20140039231A1, US20140037512A1, US20130243672A1, US20130211175A1, US20130209324A, U.S. Pat. No. 8,471,086B2. U.S. Pat. No. 8,455,708B2. U.S. Pat. No. 8,388,828B2. US20130004378A1, US20120308438A1, US20120282150A1, US20110282114A1, US20110230692A1, US20110226669A1, US20110150721A1, and U.S. Pat. No. 7,955,999B2.

In one embodiment, the apparatus is manufactured or adapted to comprise 25 to 100 wt/o of the bare metal alloy. For example, the apparatus can comprise, for example, at least 50 wt/o, or at least 70 wt % of the bare metal alloy that exhibits the low corrosion rate. In one embodiment, a previously existing apparatus using a different hydroconversion catalyst (e.g., HF, AlCl₃, or H₂SO₄) is adapted to comprise the bare metal alloy by replacing some or all of the previously existing metal or metals with the bare metal alloy.

The process for performing the hydrocarbon conversion or for handling the output of the hydrocarbon conversion uses the apparatus described above that comprises the bare metal that exhibits the low corrosion rate. The use of the apparatus can be over a broad temperature range, such as from −20° C. to 400° C. In one embodiment, the using of the apparatus comprising the bare metal alloy is performed at a temperature from 0° C. to 204° C.

### Metals and Metal Alloys:

Different metals and metal alloys are defined by their elemental composition. They can be defined by ASTM standards or by the unified numbering system. The unified numbering system (UNS) is an alloy designation system widely accepted in North America. It consists of a prefix letter and five digits designating a material composition. For example, a prefix of S indicates stainless steel alloys. C indicates copper, brass, or bronze alloys, N indicates nickel and nickel alloys, T indicates tool steels, and so on. The first 3 digits often match older 3-digit numbering systems, while the last 2 digits indicate more modern variations. ASTM E527-12 is the Standard Practice for Numbering Metals and Alloys in the Unified Numbering System (UNS).

The UNS is managed jointly by the ASTM International and SAE International. A UNS number alone does not constitute a full material specification because it establishes no requirements for material properties, heat treatment, form, or quality.

TABLE 1
Common carbon steel specifications and grades (all values in weight percent):

| ASTM Alloy and Grade | C | Mn | P | S | Cu | |
|---|---|---|---|---|---|---|
| ASTM A53: Grade A/B | 0.30 | 1.20 max | 0.05 max | 0.045 max | 0.40 | max |
| ASTM A106: Grade A/B/C | 0.35 max | 0.27-1.35 | 0.035 max | 0.035 max | 0.40 | max |
| ASTM A36 | 0.29 max | 0.80-1.35 | 0.04 max | 0.05 max | 0.20 | min[1] |

| | | | | | |
|---|---|---|---|---|---|
| ASTM A179 | 0.06-0.18 | 0.27-0.63 | 0.035 max | 0.035 max | — |
| ASTM A209 | 0.10-0.25 | 0.30-0.80 | 0.025 max | 0.025 max | — |

| ASTM Alloy and Grade | Ni | Cr | Mo | V | Si | Fe |
|---|---|---|---|---|---|---|
| ASTM A53: Grade A/B | 0.40 max | 0.40 max | 0.15 max | 0.08 max | — | Balance |
| ASTM A106: Grade A/B/C | 0.40 max | 0.40 max | 0.15 max | 0.08 max | 0.10 min | Balance |
| ASTM A36 | — | — | — | — | 0.40 max | Balance |
| ASTM A179 | — | — | — | — | — | Balance |
| ASTM A209 | — | — | — | — | — | Balance |

[1]When specified
ASTM A53, “Pipe, Steel, Black and Hot-Dipped, Zinc Coated, Welded and Seamless”
ASTM A106, “Seamless Pipe for High Temperature Service”
ASTM A36, “Carbon Structural Steel”
ASTM A179, “Seamless Cold-Drawn Low-Carbon Steel Heat-Exchanger and Condenser Tubes”
ASTM A209, “Seamless Carbon-Molybdenum Alloy-Steel Boiler and Superheater Tubes”

TABLE 2
Austenitic Stainless Steel Chemical Composition Ranges (all values in weight percent):

| Alloy | UNS # | Ni | Cr | Mo | Fe | Cu | S |
|---|---|---|---|---|---|---|---|
| 20 | N08020 | 32-38 | 19-21 | 2-3 | Bal: 32-44 | 3.0-4.0 | 0.035 |
| | | | | | | | max |
| 304L | S30403 | 8-12 | 18-20 | — | Bal: 65-74 | — | 0.03 max |
| 316L | S31603 | 10-14 | 16-18 | 2-3 | Bal: 62-72 | — | 0.03 max |
| 317L | S31703 | 11-15 | 18-20 | 3-4 | Bal: 58-68 | — | 0.03 max |
| 904L | N08904 | 23-28 | 19-23 | 4-5 | Bal: 38.8-53 | 1-2 | 0.035 |
| | | | | | | | max |
| 254SMO | S31254 | 17.5-18.5 | 19.5-20.5 | 6.0-6.5 | Bal: 51.8-57 | 0.5-1.0 | 0.01 max |
| Al6XN | N08367 | 23.5-25.5 | 20-22 | 6-7 | Bal: 41.4-50.3 | 0.75 max | 0.03 max |
| 825 | N08225 | 38-46 | 19.3-23.5 | 2.3-3.5 | Bal: 22-38 | 1.5-3.0 | 0.03 max |

| Alloy | UNS # | C | Mn | S | Ti | Al | N | P |
|---|---|---|---|---|---|---|---|---|
| 20 | N08020 | 0.07 | 2.0 max | 0.035 | — | — | — | 0.045 |
| | | max | | max | | | | max |
| 304L | S30403 | 0.03 | 2.0 max | 0.03 max | — | — | 0.1 max | 0.045 |
| | | max | | | | | | max |
| 316L | S31603 | 0.03 | 2.0 max | 0.03 max | — | — | 0.1 max | 0.045 |
| | | max | | | | | | max |
| 317L | S31703 | 0.03 | 2.0 max | 0.03 max | — | — | 0.1 max | 0.045 |
| | | max | | | | | | max |
| 904L | N08904 | 0.02 | 2.0 max | 0.035 | — | — | — | 0.045 |
| | | max | | max | | | | max |
| 254SMO | S31254 | 0.02 | 1.0 max | 0.01 max | — | — | 0.18-0.22 | 0.03 max |
| | | max | | | | | | |
| Al6XN | N08367 | 0.03 | 2.0 max | 0.03 max | — | — | 0.18-0.25 | 0.040 |
| | | max | | | | | | max |
| 825 | N08225 | 0.05 | 1.0 max | 0.03 max | 0.6-1.2 | 0.2 max | — | — |
| | | max | | | | | | |

254SMO ® and A16XN ® are registered trademarks of Avesta Steels & Alloys.

TABLE 3
Ferritic Stainless Steel Chemical Composition Ranges
(all values in weight percent):

| Alloy | UNS # | Ni | Cr | Mo | C | N | Mn |
|---|---|---|---|---|---|---|---|
| SEA- | S44660 | 1.0-3.5 | 25-28 | 3-4 | 0.03 | 0.04 max | 1.0 max |
| CURE | | | | | max | | |

| Alloy | UNS # | Si | P | S | Ti + Nb | Fe |
|---|---|---|---|---|---|---|
| SEA- | S44660 | 1.0 max | 0.04 max | 0.03 max | 0.02-1.00 | Balance |
| CURE | | | | | | |

SEA-CURE ® is a registered Trademark of Plymouth Tube Company.

TABLE 4
Duplex Stainless Steel Chemical Composition Ranges
(all values in weight percent):

| Alloy | UNS # | Ni | Cr | Mo | C | N | Mn |
|---|---|---|---|---|---|---|---|
| 2205 | S31803 | 4.5-6.5 | 22-23 | 3-3.5 | 0.03 max | 0.14-0.20 | 2.0 max |
| | S32205 | | | | | | |
| 2507 | S32750 | 6-8 | 24-26 | 3-5 | 0.03 max | 0.23-0.32 | 1.20 |
| | | | | | | | max |

| Alloy | UNS # | P | S | Cu | Fe |
|---|---|---|---|---|---|
| 2205 | S31803 | 0.03 max | 0.02 max | — | Balance |
| | S32205 | | | | |
| 2507 | S32750 | 0.035 max | 0.02 max | 0.50 | Balance |

TABLE 5
Nickel-Copper Alloy Chemical Composition Ranges
(all values in weight percent):

| Alloy | UNS # | Ni | Cu | Fe | Mn | Si | S | C |
|---|---|---|---|---|---|---|---|---|
| Monel ® | N04400 | 63.0 | 28-34 | 2.50 | 2.0 max | 0.024 | 0.50 | 0.30 |
| 400 | | min | | max | | max | max | max |

Monel ® is a trademark of Special Metals.

TABLE 6
Nickel Based Super Alloy Chemical Composition Ranges (all values in weight percent):

| Alloy | UNS # | Ni | Cr | Mo | Fe | W | Co |
|---|---|---|---|---|---|---|---|
| C-276 | N10276 | Balance | 14.5-16.5 | 15-17 | 4-7 | 3-4.5 | 2.5 max |
| C22 | N06022 | Balance | 20-22.5 | 12.5-14.5 | 2-6 | 2.5-3.5 | 2.5 max |
| B2 | N10665 | Balance | 1.0 max | 26-30 | 2.0 max | — | 1.0 max |
| Alloy | UNS # | Mn | C | P | Si | S | V |
| C-276 | N10276 | 1.0 max | 0.01 max | 0.04 max | 0.08 max | 0.03 max | 0.35 max |
| C22 | N06022 | 0.50 max | 0.01 max | 0.02 max | 0.08 max | 0.02 max | 0.35 max |
| B2 | N10665 | 1.0 max | 0.02 max | 0.04 max | 0.10 max | 0.03 max | — |

TABLE 7
Titanium Alloy Chemical Composition Ranges
(all values in weight percent):

| | | | | | | |
|---|---|---|---|---|---|---|
| Alloy | UNS # | C | Fe | H | N | O |
| Grade 2 | R50400 | 0.10 max | 0.3 max | 0.015 max | 0.03 max | 0.25 |
| | | | | | | max |
| Grade 7 | R52400 | 0.10 max | 0.3 max | 0.015 max | 0.03 max | 0.25 |
| | | | | | | max |
| Grade 12 | R53400 | 0.08 max | 0.3 max | 0.015 max | 0.03 max | 0.25 |
| | | | | | | max |
| Grade 16 | R52402 | 0.08 max | 0.3 max | 0.015 max | 0.03 max | 0.25 |
| | | | | | | max |

| Alloy | UNS # | Ti | Pd | Mo | Ni |
|---|---|---|---|---|---|
| Grade 2 | R50400 | Balance | — | — | — |
| Grade 7 | R52400 | Balance | 0.12-0.25 | — | — |
| Grade 12 | R53400 | Balance | — | 0.2-0.4 | 0.6-0.9 |
| Grade 16 | R52402 | Balance | 0.04-0.08 | — | — |

The elemental composition of metal alloys is measured using standard test methods suitable for determining the wt % of each element within acceptable precision and bias. For example, ASTM 1473-09 is a suitable test method for determining the chemical analysis of nickel, cobalt, and high-temperature alloys. In some embodiments, the wt % of an element in an alloy can be determined by difference, once the other elements have been determined.

### Corrosion Resistance:

Different metals have varying resistance to corrosion. The resistance to corrosion can be dependent on the type and length of service that the metal encounters in the apparatus made using the metal.

Corrosion rates of samples of metals (e.g., corrosion coupons) can be expressed as either milli-inches (mils) per year (mpy) or millimeters per year (mm/year, or mmy). To determine the corrosion rate, a weighed sample (e.g., corrosion coupon) of the metal or alloy under consideration is introduced into the process, and later removed after a reasonable time interval.

The corrosion coupon is then cleaned of all corrosion products and is reweighed. The weight loss is converted to a corrosion rate (CR), as follows:

Corrosion Rate (CR)=[Weight Loss (g)×K]/[Corrosion Coupon Density (g/cm3)×Exposed Area (A)×Exposure Time (hr)]

The constant K converts an experimental measurement with a fixed duration to a per-year-basis, and K can be varied depending on the measurement unites in the above equation, as shown in Table 8, to calculate the corrosion rate (CR) in various units.

TABLE 8

| Desired Corrosion Rate | Area Unit | |
|---|---|---|
| Unit (CR) | (A) | K |
| mils/year (mpy) | in² | 5.34 × 10⁵ |
| mils/year (mpy) | cm² | 3.45 × 10⁶ |
| millimeters/year (mmy) | cm² | 8.76 × 10⁴ |

Different ranges of relative corrosion resistance can be based on the following criteria for corrosion rates in Table 9.

TABLE 9

| Relative Corrosion Resistance | mils/year (mpy) | mm/year (mmy) |
|---|---|---|
| Outstanding | <1 | <0.02 |
| Standard Design Life | 1-5 | 0.02-0.10 |
| Excess Corrosion Allowance | 5-20 | 0.1-0.5 |
| Needed | | |
| Poor | >20 | >0.5 |

In one embodiment, the bare metal alloy is a stainless steel. Stainless steel differs from carbon steel by the amount of chromium present. Unprotected carbon steel rusts readily when exposed to air and moisture. An iron oxide film (the rust) is active and can accelerate corrosion by forming more iron oxide, and due to the greater volume of the iron oxide this tends to flake and fall away. Stainless steels contain sufficient chromium to form a passive film of chromium oxide, which prevents further surface corrosion by blocking oxygen diffusion to the steel surface and blocks corrosion from spreading into the metal's internal structure, and due to the similar size of the steel and oxide ions they bond very strongly and remain attached to the surface. Passivation of the bare metal alloy typically occurs when the proportion of chromium is high enough and oxygen is present.

In one embodiment, the bare metal alloy is an austenitic stainless steel. Austenitic stainless steel has austenite as its primary phase (face centered cubic crystal). Austenitic stainless steel alloys contain chromium and nickel, and sometimes molybdenum, nitrogen, or other elements. When stainless steel is exposed to temperatures from 912 to 1,394° C. (1,674 to 2,541° F.) the alpha iron in the steel undergoes a phase transition from body-centered cubic (BCC) to the face-centered cubic (FCC) configuration of gamma iron, also called austenite.

In one embodiment, the bare metal alloy has resistance to chloride stress corrosion cracking. For example, the bare metal alloy can contain a higher proportion of nickel, say from 35 to 49 wt % nickel, which can provide the bare metal alloy with resistance to chloride stress corrosion cracking. In another embodiment, the bare metal alloy comprises at least 45 wt % metals other than iron.

In one embodiment, the bare metal alloy comprises from 1.0 to 2.95 wt % copper. Examples of these types of bare metal alloys include 825 and 904L. In one embodiment, the bare metal alloy additionally comprises 5 to 25 wt % chromium. In another embodiment, the bare metal alloy additionally comprises from 0.4 to 1.4 wt % titanium.

In one embodiment, the bare metal alloy has a UNS number selected from the group consisting of N08904, S31254, N08367, and N08225.

In one embodiment, the bare metal alloy exhibits a corrosion rate from 0.001 to 0.0699 mm/year when performing the hydrocarbon conversion or handling the output of the hydrocarbon conversion. At these levels of corrosion, the apparatus can provide a standard design life or even an extended design life. In one embodiment, the bare metal alloy can be in contact with the acidic ionic liquid from 2,500 hours to 300,000 hours (or 5,000 to 220,000 hours) and remain suitable for service without excessive corrosion.

In one embodiment, the apparatus can comprise the bare metal alloy as described herein in addition to a titanium alloy. Titanium alloys comprise at least 95 wt % titanium. Representative examples of titanium alloys that can be used in the apparatus are Grade 2, Grade 7, Grade 12, and Grade 16. Titanium alloys have also been shown to give improved or comparable corrosion rates (<0.03 mm/year) compared to high-nickel alloys such as Monel® 400 and HASTELLOY® C-276 Alloy. HASTELLOY® is a registered trademark of Haynes International, Inc.

In one embodiment, additional corrosion resistance can be provided by coating the bare metal alloy in the apparatus with non-metallic materials. Examples of non-metallic materials that could be used for coating the bare metal alloy include ceramics, refractory materials, graphite, glass, and polymers. In one embodiment, the non-metallic material is an oxidic material, such as an oxide of silicon, with or without boron. Examples of polymers include polyolefins such as polypropylene and polyethylene, fluorinated polymers such as polytetrafluoroethylene, polyvinylidenefluoride and polyperfluoropropylvinylether, polymers containing sulfur and/or aromatics such as polysulfones or polysulfides, resins such as epoxy resins, phenolic resins, vinyl ester resins, furan resins. The use of polymer coatings is described in US Patent Publication No. 20140018590A1.

Acidic Ionic Liquids:

Acidic ionic liquids can be used as catalysts for various types of hydrocarbon conversions. Examples of these hydrocarbon conversions include: alkylation, isomerization, hydrocracking, polymerization, dimerization, oligomerization, acylation, metathesis, copolymerization, hydroformylation, dehalogenation, dehydration, and combinations thereof. In one embodiment the hydrocarbon conversion is alkylation of paraffins with olefins. Examples of ionic liquid catalysts and their use for alkylation of paraffins with olefins are taught, for example, in U.S. Pat. Nos. 7,432,408 and 7,432,409, 7,285,698, and U.S. patent application Ser. No. 12/184,069, filed Jul. 31, 2008. In one embodiment, the acidic ionic liquid is a composite ionic liquid catalyst, wherein the cations come from a hydrohalide of an alkyl-containing amine or pyridine, and the anions are composite coordinate anions coming from two or more metal compounds. In another embodiment the conversion of a hydrocarbon is alkylation of paraffins, alkylation of aromatics, or combinations thereof.

The most common acidic ionic liquids are those prepared from organic-based cations and inorganic or organic anions. Ionic liquid catalysts are used in a wide variety of reactions, including Friedel-Crafts reactions.

The acidic ionic liquid is composed of at least two components which form a complex. The acidic ionic liquid comprises a first component and a second component. The first component of the acidic ionic liquid will typically comprise a Lewis acid compound selected from components such as Lewis acid compounds of Group 13 metals, including aluminum halides, alkyl aluminum dihalides, gallium halide, and alkyl gallium halide (see International Union of Pure and Applied Chemistry (IUPAC), version3, October 2005, for Group 13 metals of the periodic table). Other Lewis acid compounds besides those of Group 13 metals may also be used. In one embodiment the first component is aluminum halide or alkyl aluminum dihalide. For example, aluminum trichloride (AlCl3) may be used as the first component for preparing the ionic liquid catalyst. In one embodiment, the alkyl aluminum dihalides that can be used can have the general formula AI2X4R2, where each X represents a halogen, selected for example from chlorine and bromine, each R represents a hydrocarbyl group comprising 1 to 12 atoms of carbon, aromatic or aliphatic, with a branched or a linear chain. Examples of alkyl aluminum dihalides include dichloromethylaluminum, dibromomethylaluminum, dichloroethylaluminum, dibromoethylaluminum, dichloro n-hexylaluminum, dichloroisobutylaluminum, either used separately or combined.

The second component making up the acidic ionic liquid is an organic salt or mixture of salts. These salts may be characterized by the general formula Q+A−, wherein Q+ is an ammonium, phosphonium, boronium, oxonium, iodonium, or sulfonium cation and A− is a negatively charged ion such as Cl⁻, Br⁻, ClO₄⁻, NO₃⁻, BF₄⁻, BCl₄⁻, PF₆, SbF₆⁻, AlCl₄⁻, Al₂Cl₇⁻, Al₃Cl₁₀⁻, GaCl₄⁻, Ga₂Cl₇⁻, Ga₃Cl₁₀⁻, AsF₆⁻, TaF₆⁻, CuCl₂⁻, FeCl₃⁻, AlBr₄⁻, Al₂Br₇⁻, Al₃Br₁₀⁻, SO₃CF₃⁻, and 3-sulfurtrioxyphenyl. In one embodiment the second component is selected from those having quaternary ammonium halides containing one or more alkyl moieties having from about 1 to about 9 carbon atoms, such as, for example, trimethylammonium hydrochloride, methyltributylammonium, 1-butyl pyridinium, or alkyl substituted imidazolium halides, such as for example, 1-ethyl-3-methyl-imidazolium chloride.

In one embodiment, the second component is selected from those having quaternary phosphonium halides containing one or more alkyl moieties having from 1 to 12 carbon atoms, such as, for example, trialkyphosphonium hydrochloride, tetraalkylphosphonium chlorides, and methyltrialkyphosphonium halide.

In one embodiment, the acidic ionic liquid comprises a unsubstituted or partly alkylated ammonium ion.

In one embodiment, the acidic ionic liquid is chloroaluminate or a bromoaluminate. In one embodiment the acidic ionic liquid is a quaternary ammonium chloroaluminate ionic liquid having the general formula RR′ R″ N H+Al₂Cl₇—, wherein R, R′, and R″ are alkyl groups containing 1 to 12 carbons. Examples of quaternary ammonium chloroaluminate ionic liquids are an N-alkyl-pyridinium chloroaluminate, an N-alkyl-alkylpyridinium chloroaluminate, a pyridinium hydrogen chloroaluminate, an alkyl pyridinium hydrogen chloroaluminate, a di alkyl-imidazolium chloroaluminate, a tetra-alkyl-ammonium chloroaluminate, a tri-alkyl-ammonium hydrogen chloroaluminate, or a mixture thereof.

The presence of the first component should give the acidic ionic liquid a Lewis or Franklin acidic character. Generally, the greater the mole ratio of the first component to the second component, the greater is the acidity of the acidic ionic liquid.

For example, a typical reaction mixture to prepare n-butyl pyridinium chloroaluminate ionic liquid is shown below:

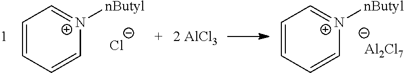

In one embodiment, the acidic ionic liquid comprises a monovalent cation selected from the group consisting of a pyridinium ion, an imidazolium ion, a pyridazinium ion, a pyrazolium ion, an imidazolinium ion, a imidazolidinium ion, a phosphonium ion, and mixtures thereof.

In one embodiment, the hydrocarbon conversion utilizes a co-catalyst to provide enhanced or improved catalytic activity. A co-catalyst can comprise, for example, anhydrous HCl or organic chloride (see, e.g., U.S. Pat. No. 7,495,144 to Elomari, and U.S. Pat. No. 7,531,707 to Harris et al.) When organic chloride is used as the co-catalyst with the acidic ionic liquid, HCl may be formed in situ in the apparatus either during the hydrocarbon conversion process or during post-processing of the output of the hydrocarbon conversion.

Acidic ionic liquid catalyzed hydrocarbon conversion products and the other outputs from the hydrocarbon conversion can include one or more halogenated components, as disclosed in U.S. Pat. No. 8,586,812. Halogenated components and HCl can contribute to excess corrosion of apparatuses used with acidic ionic liquids, including those using co-catalysts, if optimal bare metal alloys are not employed. In one embodiment, the hydrocarbon conversion is conducted in the presence of a hydrogen halide, e.g., HCl.

### Feeds for the Hydrocarbon Conversion

In one embodiment, the feed to the hydrocarbon conversion comprises at least one olefin and at least one isoparaffin. For example the feed can comprise a mixture of at least one mostly linear olefin from C2 to about C30. In another embodiment, the feed can comprise at least 50% of a single alpha olefin species. In one embodiment, the olefin feed comprises at least one isomerized olefin.

In one embodiment, the feed to the hydrocarbon conversion comprises isobutane. Isopentanes, isohexanes, isoheptanes, and other higher isoparaffins up to about C30 are also useable in the process and apparatuses disclosed herein. Mixtures of light isoparaffins can also be used in the present invention. Mixtures such as C3-C4, C3-C5, or C4-C5 isoparaffins can also be used and may be advantaged because of reduced separation costs. The feed to the hydrocarbon conversion can also contain diluents such as normal paraffins. This can be a cost savings by reducing the cost of separating isoparaffins from close boiling paraffins. In one embodiment, the normal paraffins will tend to be unreactive diluents in the hydrocarbon conversion.

### EXAMPLES

### Example 1

### Ionic Liquid Catalyst Comprising Anhydrous Metal Halide

Various acidic ionic liquid catalysts comprising a metal halide, such as AlCl₃, AlBr₃, GaCl₃, GaBr₃, InCl₃, and InBr₃, can be used for hydrocarbon conversion. In our examples, we used N-butylpyridinium chloroaluminate (C₅H₅NC₄H₉Al₂Cl₇) ionic liquid catalyst, which was made using AlCl₃. This acidic ionic liquid catalyst had the following elemental composition:

TABLE 9

| Element | Wt % |
|---|---|
| Aluminum | 12.4 |
| Chlorine | 56.5 |
| Carbon | 24.6 |
| Hydrogen | 3.2 |
| Nitrogen | 3.3 |

### Example 2

### Alkylation of C₃-C₄ Olefin and Isobutane to Make Alkylate Gasoline

A refinery isobutane stream containing 85 wt % isobutane and 15 wt % n-butane was dried with 13X molecular sieve. A refinery olefin stream containing C₃ and C₄ olefins (also referred to as C₃-C₄ Olefin) from a Fluid Catalytic Cracking Unit (FCC unit) was dried with 13X molecular sieve and isomerized with a Pd/Al₂O₃ catalyst at 150° F. and 250 psig (1724 kPa) in the presence of hydrogen to produce isomerized C₃ and C₄ olefin feed with the molecular composition shown in Table 10.

TABLE 10

| Molecule | Mol % |
|---|---|
| Propane, C3 | 13.3 |
| Propylene, C3 = | 25.4 |
| 1-Butene, 1-C4 = | 2.3 |
| 2-Butene, 2-C4 = | 16.2 |
| Isobutylene, i-C4 = | 6.7 |
| n-Butane, nC4 | 12.4 |
| Isobutane, iC4 | 22.2 |
| C5+ | 1.6 |
| Sum | 100.0 |

Evaluation of the alkylation of the isomerized C₃ and C₄ olefin feed with the dried refinery isobutane stream was performed in a continuously stirred tank reactor. A 9:1 molar mixture of the dried refinery isobutane stream and the isomerized C₃ and C₄ olefin feed was fed to the reactor while vigorously stirring. The ionic liquid catalyst described in Example 1 was fed to the reactor via a second inlet port targeted to occupy 6 vol % in the reactor. A small amount of n-butyl chloride was added to produce anhydrous HCl gas in situ. The average residence time in the reactor for the combined volume of feeds and catalyst was about 12 minutes. The reactor outlet pressure was maintained at 200 psig (1379 kPa) and the reactor temperature was maintained at 95° F. (35° C.) using external cooling.

A corrosion coupon holder was installed right after the reactor and reactor effluent flowed through the coupon holder. The corrosion coupon holder was operated at approximately 95° F. (approximately 35 degree Celsius) and around 200 psig (1379 kPa) pressure. The corrosion coupon holder was a completely liquid filled unit with a mixture of ionic liquid catalyst, propane, isobutane, n-butane, and alkylate product. The corrosion coupon holder was designed to hold 12 to 15 corrosion coupons made of various materials. Each corrosion coupon was separated from the others using Teflon spacers.

The reactor effluent, after passing through the corrosion coupon holder, was separated using a coalescing separator into a hydrocarbon phase and an ionic liquid catalyst phase. The hydrocarbon phase was further separated using three distillation columns into multiple streams, including: a gas stream containing a C₃⁻ fraction, an nC₄ stream, an iC₄ stream, and an alkylate stream. The ionic liquid catalyst was recycled back to the alkylation reactor for repeated use. To maintain the activity of the ionic liquid catalyst, a fraction of the used ionic liquid catalyst was sent to a regeneration reactor that reduced the level of conjunct polymer in the ionic liquid catalyst. The level of the conjunct polymer was maintained from 2 to 5 wt % and alkylate gasoline with good properties was continuously produced during the course of these alkylation experiments.

After 1 to 34 months of operation, the corrosion coupons were removed from the corrosion coupon holder, rinsed with methanol, and dried. The dried corrosion coupons were weighed, and based on the coupon weight losses and times in service, the corrosion rates were calculated. The experiments were repeated several times to generate statistically significant results.

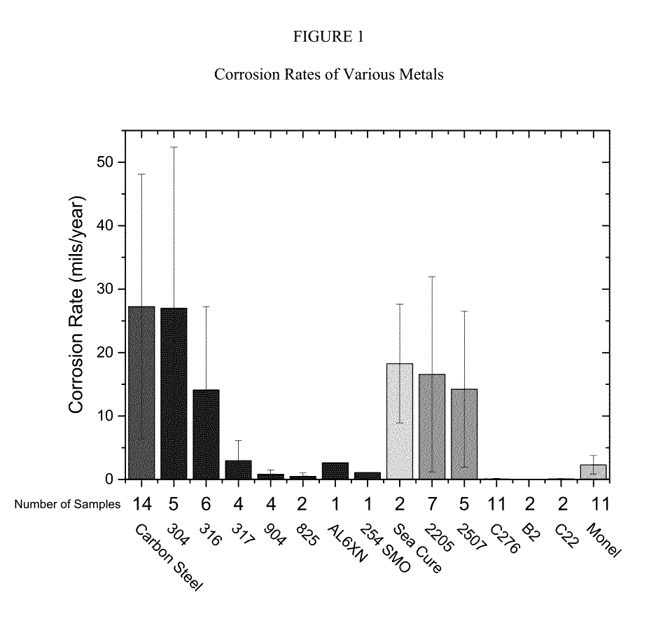

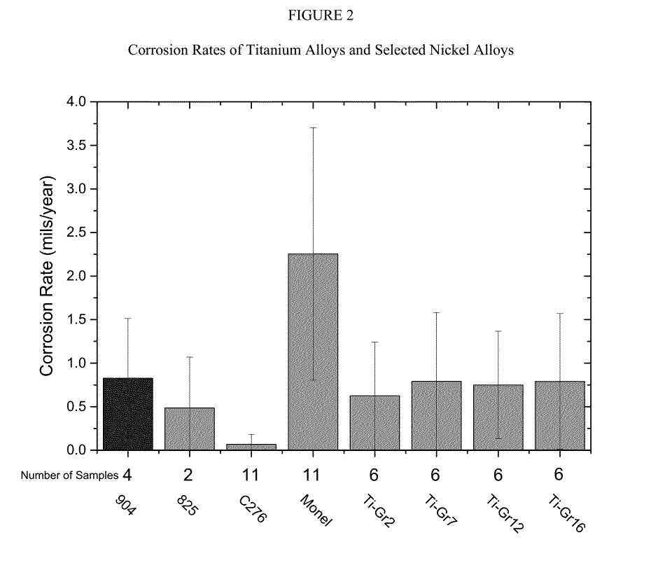

The results of these experiments are shown in FIGS. 1 and 2. Error bars (1 standard deviation) are shown in the figures.

### Example 3

### Corrosion Rating of Alloys

The typical chemical compositions and relative corrosion resistance obtained in the alloys tested in the experiments described in Example 2 are summarized in Table 11, below.

TABLE 11

| | Typical Chemical Composition of Alloy | | | | | | | | | | Corrosion Rate, | Corrosion Rate, | |
|---|---|---|---|---|---|---|---|---|---|---|---|---|---|
| Alloy | Ni | Cr | Mo | Cu | W | Fe | N | C | Ti | Other | mpy | mm/year | Corrosion Rating |
| C-276 | 58 | 16 | 16 | — | 3.5 | 5.5 | — | — | — | | 0.1 | 0.0025 | Outstanding |
| C22 | 58 | 22 | 13 | — | 3.2 | 3 | — | — | — | | 0.1 | 0.0025 | Outstanding |
| B2 | 68 | 1 | 28 | — | — | 2 | — | 0.02 | — | | 0.0 | 0.000 | Outstanding |
| Monel ® | 66 | — | — | 31 | — | 2.5 | — | 0.3 | — | | 2.3 | 0.0584 | Standard design |
| 400 | | | | | | | | | | | | | life |
| 825 | 42 | 21 | 3 | 2 | — | ~30 | — | <0.05 | 1 | Mn: <1.0 | 0.5 | 0.0127 | Outstanding |
| | | | | | | | | | | Si: <0.5 | | | |
| 904L | 26 | 21 | 4 | 1 | | ~40 | — | <0.02 | — | Mn: <2.0 | 0.8 | 0.0203 | Outstanding |
| | | | | | | | | | | Si: <1.0 | | | |
| AL6XN | 24 | 20 | 6 | 0.1 | — | ~48 | 0.2 | 0.02 | — | Mn: <2.0 | 2.6 | 0.0660 | Standard design |
| | | | | | | | | | | Si: <1.0 | | | life |
| 254SMO | 18 | 20 | 6 | 0.7 | — | ~54 | 0.2 | <0.02 | — | Mn: <1.0 | 1.1 | 0.0279 | Standard design |
| | | | | | | | | | | Si: <0.8 | | | life |
| 317L | 13 | 18 | 3 | — | — | ~60 | 0.1 | <0.03 | — | Mn: <2.0 | 2.9 | 0.0737 | Excess corrosion |
| | | | | | | | | | | Si: <0.75 | | | allowance needed |
| 316L | 12 | 16 | 2 | — | — | ~65 | 0.1 | <0.03 | — | Mn: <2.0 | 14.1 | 0.3581 | Excess corrosion |
| | | | | | | | | | | Si: <0.75 | | | allowance needed |
| 304L | 8 | 18 | — | — | — | 65+ | — | <0.03 | — | Mn: <2.0 | 27.0 | 0.6858 | Poor |
| | | | | | | | | | | Si: <0.75 | | | |
| Sea | 2 | 28 | 4 | | | ~47 | <0.04 | <0.03 | — | Ti + Nb: | 18.3 | 0.4648 | Excess corrosion |
| Cure | | | | | | | | | | 0.02-1.0 | | | allowance needed |
| 2205 | 5 | 22 | 3.2 | | | ~60 | 0.18 | <0.03 | — | Mn: 1.5 | 16.6 | 0.4216 | Excess corrosion |
| | | | | | | | | | | | | | allowance needed |
| 2507 | 7 | 25 | 4 | <0.5 | | ~49 | 0.3 | <0.03 | — | Mn: 1.0 | 14.2 | 0.3607 | Excess corrosion |
| | | | | | | | | | | | | | allowance needed |
| Carbon | — | — | — | — | — | ~99 | | 0.20 | — | Mn: 0.8 | 27.2 | 0.6909 | Poor |
| Steel | | | | | | | | | | | | | |
| Ti-Gr2 | — | — | — | — | — | 0.2 | 0.03 | 0.08 | 99+ | Pd: 0.15 | 0.6 | 0.0152 | Outstanding |
| Ti-Gr7 | — | — | — | — | — | 0.25 | 0.05 | 0.06 | 99+ | | 0.8 | 0.0203 | Outstanding |
| Ti-Gr12 | 0.8 | — | 0.3 | — | — | <0.30 | <0.03 | <0.08 | 98+ | | 0.7 | 0.0178 | Outstanding |
| Ti-Gr16 | | | | | | <0.30 | <0.03 | <0.08 | 99+ | Pd: 0.06 | 0.8 | 0.0203 | Outstanding |

Alloy 825 and alloy 904L had lower Ni contents than alloys C-276, C22, B2, and Monel® 400, while alloy 825 and alloy 904L exhibited comparable corrosion ratings. Alloys 825 and 904L are both moderately priced and can provide improved value for applications using an acidic ionic liquid. However, the lower Ni content of the 904L could lead to susceptibility to chloride stress corrosion cracking during operation and steam cleaning while the Ni content of Alloy 825 is closer to the range desired for providing acceptable resistance to chloride stress corrosion cracking of austenitic stainless steel alloys.

All four of the titanium alloys tested gave outstanding corrosion ratings as well, and they are also moderately priced and readily available.

### Example 4

### Analysis of Alloy Elements for Corrosion Rates

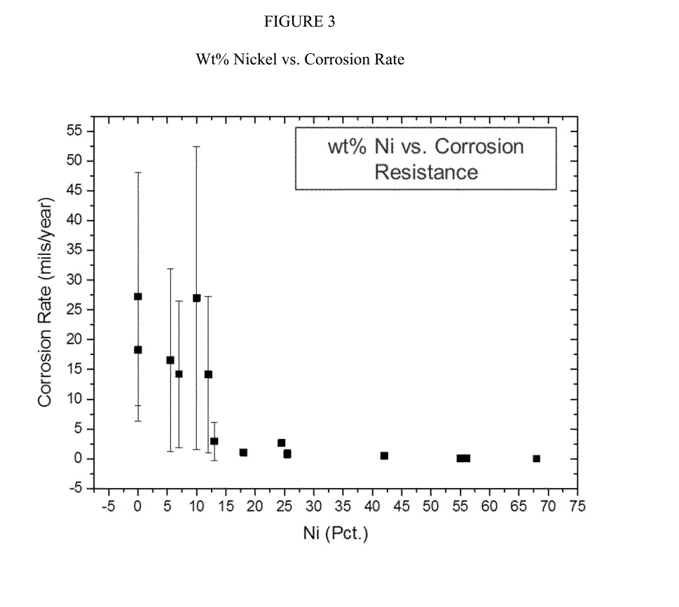

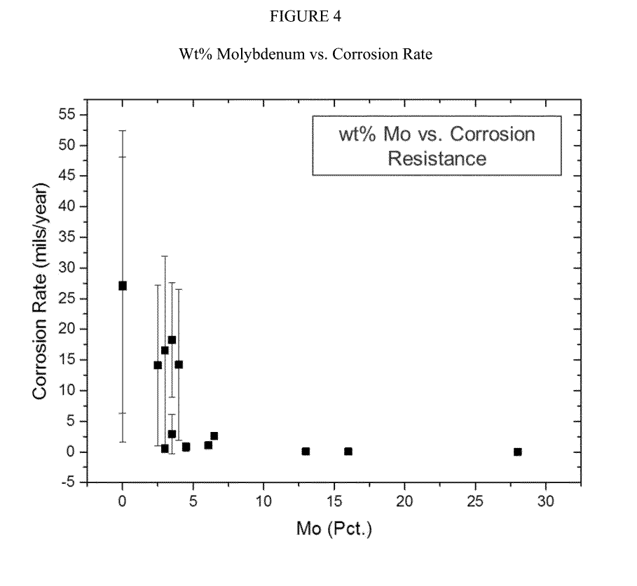

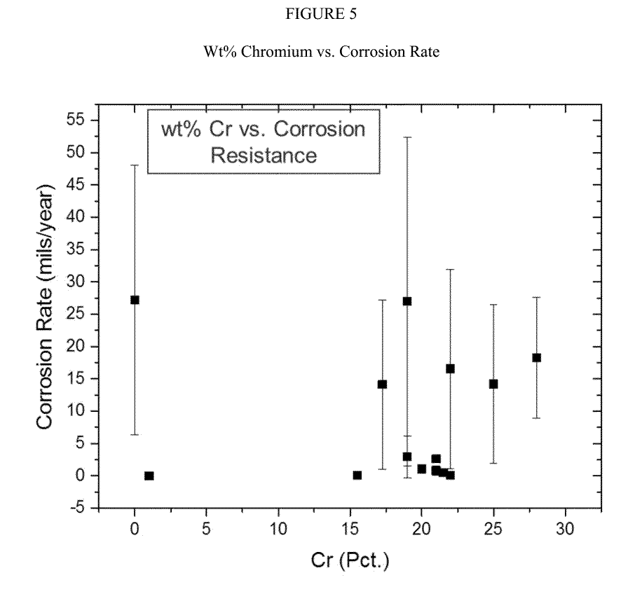

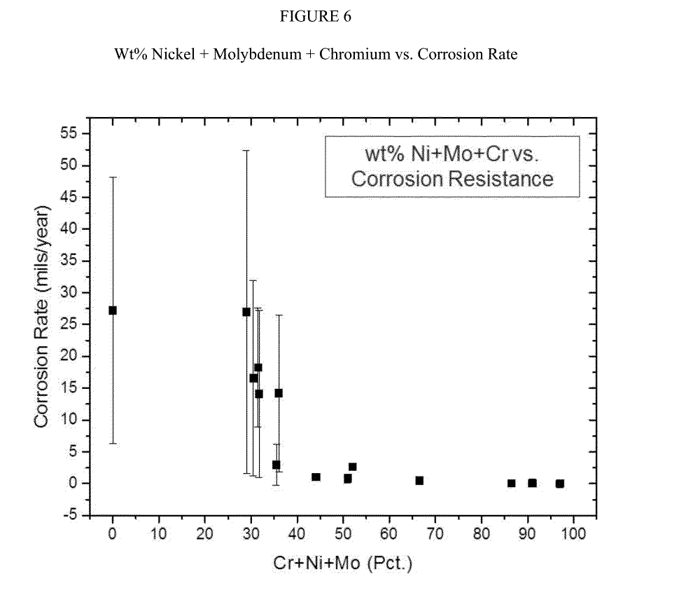

Using the data collected in the alkylation experiments conducted in Example 2, the weight percent of the different metals in the corrosion coupons were plotted versus the corrosion rates (mils/year) that were obtained. The results obtained for nickel are shown in FIG. 3. The results obtained for molybdenum are shown in FIG. 4. The results obtained for chromium are shown in FIG. 5. The results obtained for the total weight percent of chromium, nickel, and molybdenum are shown in FIG. 6.

The corrosion rate results summarized in FIGS. 3-6 indicated that a minimum nickel content of about 15 wt % and a minimum molybdenum content of about 2.5 wt % were optimal to provide corrosion resistance to metal alloys comprising iron that were used for catalysis using an ionic liquid catalyst. A similar threshold value for chromium to provide corrosion resistance was not observed.

The results summarized in FIG. 2 indicated that either pure titanium or titanium alloys were highly corrosion resistant in our alkylation experiments using an ionic liquid catalyst. The titanium corrosion rates were somewhat higher than the corrosion rates we obtained with C-276 alloy. The titanium corrosion rates were significantly lower than the corrosion rate for Monel® 400, and the corrosion rates were comparable to super austenitic stainless steels (904 and 825). The corrosion rates were not significantly different between the different titanium alloys.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed. Unless otherwise specified, all percentages are in weight percent.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

The invention illustratively disclosed herein suitably may be practiced in the absence of any element which is not specifically disclosed herein.

What is claimed is:

1. An apparatus configured for performing a hydrocarbon conversion using an acidic ionic liquid that comprises a metal halide, or for handling of an output of the hydrocarbon conversion, comprising: a bare metal alloy, wherein the bare metal alloy comprises:
from 15.1 to 49 wt % nickel, from 2.3 to 10 wt % molybdenum, from 0.00 to 2.95 wt % copper, and 20 to 59 wt % iron; and
wherein the bare metal alloy exhibits a corrosion rate less than 0.07 mm/year when performing the hydrocarbon conversion or handling the output of the hydrocarbon conversion.

2. The apparatus of claim 1, wherein the bare metal alloy is an austenitic stainless steel.

3. The apparatus of claim 1, wherein the bare metal alloy comprises at least 35 wt % nickel and has resistance to chloride stress corrosion cracking.

4. The apparatus of claim 1, wherein the apparatus is selected from the group consisting of a reactor, a conduit, a fitting, a heat exchanger, a phase separator, a distillation unit, and combinations thereof.

5. The apparatus of claim 1, wherein the apparatus is configured to produce an alkylate gasoline blending component, a distillate fuel, a base oil, or combinations thereof.

6. The apparatus of claim 1, wherein the apparatus is manufactured or adapted to comprise at least 70 wt % of the bare metal alloy.

7. The apparatus of claim 1, wherein the bare metal alloy additionally comprises from 5 to 25 wt % chromium.

8. The apparatus of claim 1, wherein the bare metal alloy additionally comprises from 0.4 to 1.4 wt % titanium.

9. The apparatus of claim 1, wherein the bare metal alloy comprises from 1.0 to 2.95 wt % copper.

10. The apparatus of claim 1, wherein the bare metal alloy has a UNS number selected from the group consisting of N08904, S31254, N08367, and N08225.

11. The apparatus of claim 1, wherein the bare metal alloy comprises at least 45 wt % metals other than iron.

12. A process for performing a hydrocarbon conversion using an acidic ionic liquid that comprises a metal halide, or for handling of an output of the hydrocarbon conversion, comprising: using an apparatus comprising a bare metal alloy, wherein the bare metal alloy comprises:
from 15.1 to 49 wt % nickel, from 2.3 to 10 wt % molybdenum, from 0.00 to 2.95 wt % copper, and 20 to 59 wt % iron; and
wherein the bare metal alloy exhibits a corrosion rate less than 0.07 mm/year when performing the hydrocarbon conversion or handling the output of the hydrocarbon conversion.

13. The process of claim 12, wherein the hydrocarbon conversion is selected from the group consisting of an alkylation, a polymerization, a dimerization, an oligomerization, an acylation, a hydrocracking, a metathesis, a copolymerization, an isomerization, a carbonylation, a hydroformylation, a dehalogenation, a dehydration, and combinations thereof.

14. The process of claim 12, wherein the bare metal alloy is in contact with the acidic ionic liquid for 5,000 to 220,000 hours.

15. (canceled)

16. The process of claim 12, wherein the acidic ionic liquid is a chloroaluminate or a bromoaluminate.

17. The process of claim 12, wherein the acidic ionic liquid comprises a monovalent cation selected from the group consisting of a pyridinium ion, an imidazolium ion, a pyridazinium ion, a pyrazolium ion, an imidazolinium ion, a imidazolidinium ion, a phosphonium ion, an ammonium ion, and mixtures thereof.

18. The process of claim 12, wherein the acidic ionic liquid comprises an unsubstituted or partly alkylated ammonium ion.

19. The process of claim 12, wherein the using the apparatus comprising the bare metal alloy is performed at a temperature from 0° C. to 204° C.

20. The process of claim 12, wherein the hydrocarbon conversion is conducted in a presence of a hydrogen halide.
